(19) United States
(12) Patent Application Publication
(10) Pub. No.: US 2019/0054020 A1
(43) Pub. Date: Feb. 21, 2019

(54) ANESTHETIC CHEWING GUM

(71) Applicant: MANAL MANSOUR ABU AL-MELH, SAFAT (KW)

(72) Inventor: MANAL MANSOUR ABU AL-MELH, SAFAT (KW)

(21) Appl. No.: 15/680,113

(22) Filed: Aug. 17, 2017

(51) Int. Cl. A61K 9/68; A61K 31/167; A61K 9/20

(52) U.S. Cl. CPC A61K 9/0058; A61K 31/167; A61K 9/2095; A61K 9/2013; A61K 9/2068; A61K 9/2009

(57) ABSTRACT

The anesthetic chewing gum includes lidocaine HCL and prilocaine HCL as the anesthetic ingredients, as well as a chewing gum base. The anesthetic chewing gum may also include one or more sweeters, an anti-adherent, a lubricant, an opacifier, a glidant, a flavoring agent, and a flavor enhancer.

### BACKGROUND

### 1. Field

The present disclosure relates to oral pain relief, and particularly to an anesthetic chewing gum to help alleviate pain from orthodontic procedures, such as orthodontic elastomeric separator placement.

### 2. Description of the Related Art

Orthodontic elastomeric separator placement is generally associated with tenderness and pain for almost all patients. Tooth separation can have an undesirable impact on a patient's chewing, daily activity, and sleep. Such adverse impacts can affect the patient's acceptance and compliance to this or other orthodontic treatments.

Various techniques currently exist for controlling or managing the pain associated with dental procedures, including using ibuprofen, topical anesthetics, medicated wax containing benzocaine for relieving mucosal discomfort from the application of orthodontic appliances, as well as chewing a plastic wafer (i.e. viscoelastic bite wafers) during the first few hours after the dental appliance has been implanted. These techniques generally do not provide pain relief for prolonged periods of time.

Thus, an anesthetic chewing gum solving the aforementioned problems is desired.

### SUMMARY

The anesthetic chewing gum includes lidocaine HCL and prilocaine HCL as the anesthetic ingredients, as well as a chewing gum base. The anesthetic chewing gum may also include one or more sweeteners, an anti-adherent, a lubricant, an opacifier, a glidant, a flavoring agent, and a flavor enhancer.

These and other features of the present invention will become readily apparent upon further review of the following specification and drawings.

### BRIEF DESCRIPTION OF THE DRAWINGS

The sole drawing figure is a graph that illustrates the efficacy of the anesthetic chewing gum.

### DETAILED DESCRIPTION OF THE PREFERRED EMBODIMENTS

An anesthetic chewing gum (hereinafter referred to as “ACG”) can be used for soothing pain and discomfort resulting from orthodontic procedures, such as orthodontic elastomeric separator placement. The ACG can include an effective amount of one or more anesthetic drugs, a chewing gum base, and one or more flavoring agents. The anesthetic drugs can include at least one of lidocaine HCL and prilocaine HCL. Preferably, the ACG includes both lidocaine HCL and prilocaine HCL in equal amounts. The chewing gum base can be any suitable gum base, such as HEALTH IN GUM from Cafosa Health. The flavoring agents can include peppermint oil, and a flavor enhancer, such as menthol. The ACG can include one or more additional ingredients suitable for a chewing gum, including, but not limited to one or more sweeteners, such as sorbitol and/or mannitol, an anti-adherent, such as talc, a lubricant, such as magnesium stearate, an opacifier, such as titanium dioxide, and a glidant, such as colloidal silicon dioxide.

Table 1 lists the composition of an exemplary chewing gum piece or chewing gum tablet according to the present teachings.

TABLE 1

| Ingredient | Role of Ingredient | Quantity (mg/tablet) |
|---|---|---|
| Lidocaine HCl | Anesthetic drug | 2 |
| Prilocaine HCl | Anesthetic drug | 2 |
| Health In Gum | Chewing Gum Base | 436 |
| Sorbitol | Primary Sweetener | 15 |
| Mannitol | Secondary Sweetener | 15 |
| Talc | Anti-adherent | 10 |
| Magnesium Stearate | Lubricant | 5 |
| Titanium Dioxide | Opacifier | 5 |
| Colloidal Silicon Dioxide | Glidant | 2.5 |
| Peppermint Oil | Flavoring agent | 2.5 |
| Menthol | Flavor enhancer | 5 |

Both lidocaine HCL and prilocaine HCL were evaluated via UV spectrophotometry at a specific wavelength of maximum absorbance, and via infra-red spectroscopy. Lidocaine HCL and prilocaine HCL, as raw materials, have a maximum absorbance λmax of 254 nm and 240 nm, respectively.

To prepare the ACG, the lidocaine HCL, prilocaine HCL, and one or more additional ingredients can be passed through a sieve, such as a #40 sieve, prior to weighing to ensure a uniform size distribution. Once the desired amount of each ingredient has been obtained, lidocaine HCL and the prilocaine HCL are alternately combined, such as in small amounts, with each of the remaining chewing gum ingredients to ensure, a uniform, such as substantially uniform, distribution of ingredients within the ACG. For example, on a glass slab, lidocaine HCL may be mixed with an amount of anti-adherent, such as in small amounts, to form a lidocaine HCL/anti-adherent mixture. Separately and in similar amounts, prilocaine HCL may be mixed with an amount of anti-adherent to form a prilocaine HCL/anti-adherent mixture. The prilocaine HCL/anti-adherent mixture may then be combined and mixed with the lidocaine HCL/anti-adherent mixture to form a homogenized prilocaine HCL-lidocaine HCL/anti-adherent mixture. Lidocaine HCL may then be mixed with another ingredient, such as an amount of lubricant, such as in small amounts, to form a lidocaine HCL-lubricant mixture. Subsequently and in similar amounts, prilocaine HCL may be mixed with the lubricant, such as in small amounts, to form a prilocaine HCL-lubricant mixture. The prilocaine HCL-lubricant mixture may then be combined and mixed with the lidocaine HCL-lubricant mixture to form a homogenized lidocaine HCL-prilocaine HCL lubricant mixture. The homogenized lidocaine HCL-prilocaine HCL-lubricant mixture may then be mixed with the homogenized lidocaine HCL-prilocaine/anti-adherent mixture. The same procedure of alternate mixing of lidocaine HCL and prilocaine HCL may be done with each of the remaining ingredients, such as the sweeteners, the opacifier, the colloidal silicon dioxide, and the flavor enhancer to form a homogenized final mixture. The homogenized final mixture may then be mixed with the gum base, preferably in small amounts, until all the ingredients are uniformly mixed together to form a homogenized lidocaine HCL-prilocaine HCL gum base mixture. Subsequently, the flavoring agent, such as peppermint oil, can be added to the mixture, e.g., dropwise, to form granules.

The granules formed by mixing the homogenized lidocaine HCL-prilocaine HCL gum base mixture and the flavoring agent can then be kept at room temperature for about 30 minutes for drying, such as atmospheric drying, at room temperature. It is desirable to have free-flowing particles so that the particles may uniformly flow from the hopper to the die cavity of a tablet machine to produce ACG tablets of constant weight, as discussed further herein. Preferably, the angle of repose of the granules is about 26.01±0.097, for excellent flow properties of the granules. It is to be noted that the more acute the angle, the greater the flowability of the sample and vice versa. The bulk density and tap density of the granules is preferably around 0.621 g/ml and 0.636 g/ml respectively, for good flow of the granulation under gravity.

The granules can be dried and mixed within a container, such as a wide mouth container, to form a homogenized granule mixture. The homogenized granule mixture can then be used to form the ACG. The ACG may be produced in any suitable form, such as in tablet form, via a direct compression technique on a single station compression machine, such as an EREWKA single punch EP1 (machine manufactured in Frankfurt, Germany), using a set of standard 10 mm round concave punches and a die. It is feasible to directly compress the granules without any stickiness due to an appropriate combination of the synthetic gum, anti-adherent, glidant, and opacifier. It is to be noted that the ACG tablets can be packaged in any suitable manner, as well as in any suitable quantity, such as two tablets. The ACG tablets can be consumed in any suitable quantity, such as two at a time, to ensure the effectiveness of the anesthetic component of the ACG.

The ACG tablet can be an off-white color, and generally round with a smooth surface. The ACG tablet can have a diameter of about 10 mm, e.g., 10.02 mm±0.040, and a thickness of about 6 mm, e.g., 6.56 mm±0.119. At a constant hardness, tablet thickness tends to vary with changes in die fill and with particle size distribution. Further, the ACG tablets can have any suitable weight, such as a weight of about 499 mg, e.g., 499.65 mg 0.381, so as to comply with the United State Pharmacopeia (USP) weight variation requirements (NMT±5%).

The hardness e.g. crushing strength, of the ACG tablet plays a vital role in tablet production since high hardness values may increase disintegration times and decrease drug release or, on the other hand, if the hardness is too low then friability may be too high. Preferably, the average hardness of the ACG tablets is about 4.0 kg/cm², e.g., 4.03 kg/cm²±0.253.

The following examples illustrate the present teachings.

### Example 1

The preparation of local anesthetic-containing chewing gum formulation was investigated using Food and Drug (FDA) certified ingredients.

Several preliminary trials were performed to optimize the anesthetic drug content and other necessary functional excipients to prepare the chewing gum tablets by direct compression technique. Each tablet contained 2 mg of each of the two tested local anesthetics; lidocaine and prilocaine, in addition to peppermint oil as a flavoring agent.

All of the solid ingredients were passed individually through 40# sieve before weighing, in order to ensure uniform size distribution. The two anesthetic drugs were alternately mixed on a glass slab in sequence with talc, magnesium stearate, titanium dioxide, mannitol, sorbitol, menthol and Aerosil® in geometric proportion. This blend was then mixed geometrically with the gum base. Finally peppermint oil was added drop wise and mixed. The granules were kept for air drying for 30 minutes and then mixed in a wide mouth glass container before direct compression into tablets.

The identification of prilocaine HCl and lidocaine HCl in the raw materials was evaluated by UV spectrophotometry (UV-1601, UV VIS Spectrophotometer, Schimadzu, Japan) at the specific wavelength of maximum absorbance and by infra-red spectroscopy (Nicolet iS50 FTIR, Thermo Scientific, USA).

Flow properties of the granules were determined by measurement of angle of repose, bulk density and tap density.

### Example 2

The final granule blend was directly compressed into tablets on a single station compression machine (Erweka EKO, GmbH, Germany) using a set of 10 mm round standard concave punches and die.

The tablets were tested for color, taste, dimensions and freedom from any physical flaws. The dimensions of six tablets were measured using a Vernier Caliper (Draper Expert Tool Ltd., UK), and the average weight (±SD) was calculated. The tablets were off-white color, round, with a smooth surface, had a mint odor and were free from physical flaws. The taste and consistency were acceptable. The tablets had an average diameter of 10.02 mm±0.040, and a thickness of 6.56 mm±0.119. At a constant hardness, tablet thickness tends to vary with changes in die fill and with particle size distribution.

The weight of tablets was routinely measured during compression in order to ensure that each tablet contains the proper amount of drug. The weight variation test was performed by selection of twenty medicated chewing gum randomly. They were weighed individually on an electronic balance (GR 300, A&D Company, Limited, Japan) and the average weight was calculated from the total weight. Then, the percentage deviation from the average was calculated. The average weight of the tablets was 499.65 mg±0.381.

The hardness of six medicated chewing gums was determined by diametrical compression using a Monsanto hardness tester (Campbell Electronics, India), and the average (±SD) was calculated. The average hardness of the tablets was 4.03 kg/cm²±0.253.

### Example 3

A case-control study was conducted at Kuwait University, Faculty of Dentistry Dental Clinic in Kuwait. The study's drug formulation, design and protocol were approved by the Ethical Committee of the Health Sciences Center at Kuwait University.

Forty seven voluntary subjects, between 23 and 41 years of age, were approached. The subjects were undergraduate 7^{th} year dental students, academic staff members including assistant professors, and dental assistants from the Faculty of Dentistry, Kuwait University, Kuwait. The subjects' rights have been protected, and a written informed consent was obtained from each of the forty subjects participating in this study.

A person was eligible to participate if s/he fulfilled the following inclusion criteria: 1) medically healthy subjects with no history of allergies to any of the anesthetic ingredients, 2) individuals with healthy temporomandibular joints, 3) healthy gingival tissues, 4) sound and intact posterior teeth and 5) tight interproximal contact point between the right or left maxillary 1^{st} molar and 2^{nd} premolar where the orthodontic elastomeric separator will be positioned. The tightness of the contact between the 1^{st} molar and 2^{nd} premolar was checked with a piece of waxed dental floss.

Each participant was further encouraged to have a good breakfast before the start of the procedure as they, for the purpose of pain/discomfort registration, were advised to try to refrain from eating during the study except during lunch time. The individuals were allowed to drink fluids throughout the study. Pregnant woman, subjects with systemic diseases, and subjects taking systemic analgesics were excluded from this study. While forty seven individuals applied to participate, seven declined to participate, three of which were men. Thus, the study included forty females between the ages of 23 and 41 with an 85.1% response rate. Once each participant had the orthodontic elastomer separator placed, she was randomly allocated to one of the two groups: the experimental (ACG) group and the control group. The subjects were randomly selected, e.g., every second subject received the ACG. The experimental group received the ACG and the control group did not take any type of pain/discomfort relief remedy. Both groups received elastomeric separators for a total duration of eight hours.

Each participant had an orthodontic elastomeric separator (not shown) implanted for a total duration of eight hours, the elastomeric separator being removed after eight hours with an explorer. The method for the orthodontic elastomeric separator placement involved the use of two pieces of waxed dental floss. The separator was then stretched using the floss and wedged between the 2^{nd} premolar and 1^{st} molar on the right or left side of all individuals. Immediately after placement of the elastomeric separator, the subjects in both groups were instructed to register their immediate pain/discomfort response at placement of the separator (zero hour), then after one hour, after 4 hours and after eight hours from the separator placement.

Each individual in the ACG group was given a total of 36 ACG tablets and instructed to chew on the first two chewing gum tablets once the separator was placed and after recording the immediate response (zero hour). The subjects were instructed chew only on the side where the separator was placed. In this group, 18 subjects had the separator placed on the right side and 2 subjects received it on the left side.

The subjects were instructed to chew on two new gum tablets every 25 minutes for the duration of the experiment except for the lunch hour break. The subjects in each group were instructed to report their pain/discomfort response after one hour, four hours, and eight hours from placement of the elastomeric separators. After the eighth hour, the separator was removed with an explorer. None of the individuals in the ACG group were told of the ingredients of the ACG. Each individual in the ACG group was instructed to stop chewing the ACG during the one-hour lunch break and to resume chewing the ACG right after lunch.

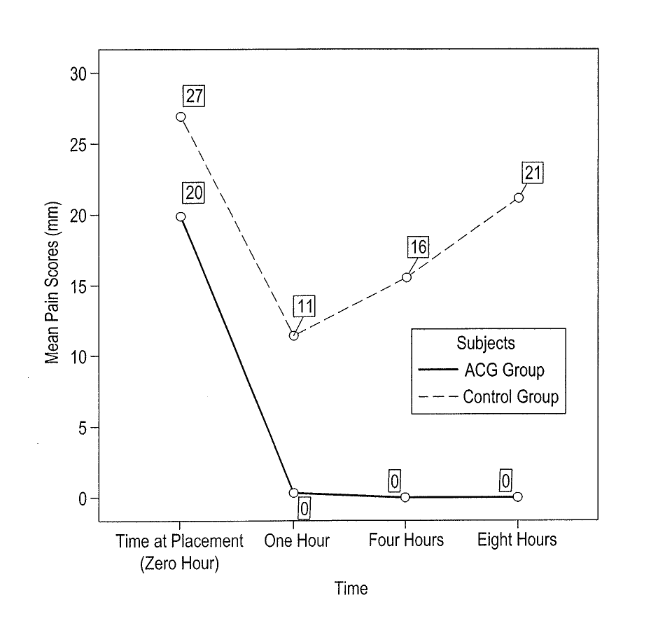

Registration of pain sensation was recorded on a Visual Analogue Scale (VAS), wherein the Mean Pain Score (mm) was recorded on the ‘Y’ axis and Time (Zero Hour, One Hour, Four Hours, and Eight Hours) was recorded on the ‘X’ axis, as illustrated on the graph 200 in FIG. 2. The overall pain perception was measured by the individuals using a scale from 0 to 100 mm horizontal non-graded VAS, wherein “0” indicated “no discomfort/pain” and “100” indicated “worst possible discomfort/pain”. The individuals in the ACG group were also interviewed regarding the effectiveness and taste of the ACG, and whether there were any local or systemic side effects experienced from the ACG and/or the orthodontic elastomeric separator. It is to be noted other than mild chewing-related soreness experienced by some participants in the ACG group, which disappeared right after the completion of the study, no harm was reported by the ACG group.

The individuals in the control group, on the other hand, were not given any type of pain remedy. The individuals were instructed not to chew gum or take any systemic analgesic. In the control group, 16 individuals received the separator on the right side and 4 individuals received the separator on the left side. The individuals in the control group were told to avoid eating during the eight-hour duration except during the one-hour lunch time. If they needed to eat, they were advised to chew on the side which did not contain the elastomeric separator. The individuals in the control group were also allowed to drink fluids until the completion of the study. Similar to the individuals in the ACG group, the individuals in the control group were instructed to report their pain/discomfort response right after separator placement (zero hour), and then, after one hour, four hours, and eight hours on the VAS. After the eighth hour, the separator was removed with an explorer. The subjects were then interviewed regarding any local adverse effects associated with the separator placement. No harm was reported by the control group.

The information from the individuals in both the ACG group and the control group was entered and analyzed using SPSS version 24 (IBM, U.S.A.). The mean (SD) and the median were used for descriptive statistics. The value of pain perception among individuals in the ACG group and individuals in the control group at different time intervals was tested for normality using the Shapiro-Wilk test that produced a p-value of <0.001 indicating a non-normal distribution. A non-parametric test, such as the Mann-Whitney U test (See Table II below), was performed to test the differences in the median of pain perception scores among those individuals in the ACG group and those individuals in the control group. A p-value of <0.05 was considered the significance level.

The pattern of pain perception of individuals in the two groups at different points of time is illustrated by the graph in the sole drawing figure. The Mean Pain Perception score showed decreasing values during the first hour of separator placement from “20 mm” to almost “0 mm” for participants in the ACG Group. Further, the Mean Pain Perception remained at the “0 mm” level in the next two measures at four and eight hours after using the ACG. The graph also demonstrates that the Mean Pain Perception score showed less decrease among individuals in the control group, such as from “27 mm” to “11 mm” after one hour, and then started to increase gradually to reach means of “16 mm” and “21 mm” at four and eight hours, respectively. The ACG significantly decreased the pain/discomfort from the orthodontic elastomeric separators during the eight hour time frame.

The median (minimum, maximum) pain perception among individuals in the ACG group and in the control group at time of placement of separators (zero hour), one, four, and eight hours is illustrated below in Table 2. The median of pain perception was higher among individuals in the control group (23.0 mm) than among those in the ACG group (16.0 mm) but this difference was not statistically significant. However, the median of pain perception was equal to “0 mm” at one hour, four hours, and eight hours after separator placement among individuals in the ACG group, which was less than that reported among individuals in the control group, which was 5.5 mm, 13.0 mm and 6.0 mm, respectively. The differences in the median of pain perception between those individuals in the ACG group and those in the control group at each of the three time points (one, four, and eight hours) were highly significant with a p-value of <0.0001.

TABLE 2

| | ACG Group | | Control Group | | |
|---|---|---|---|---|---|
| | n = 20 | | n = 20 | | p- |
| Time | Median (Min, Max) | | Median (Min, Max) | | value* |
| Time at placement | 16.0 | (2.0, 80.0) | 23.0 | (3.0, 90.0) | 0.250 |
| (zero hour) | | | | | |
| One hour | 0.0 | (0.0, 4.0) | 5.5 | (0.0, 46.0) | <0.0001 |
| Four hours | 0.0 | (0.0, 4.0) | 13.0 | (0.0, 42.0) | <0.0001 |
| Eight hours | 0.0 | (0.0, 3.0) | 6.0 | (0.0, 91.0) | <0.0001 |

It is to be understood that the present invention is not limited to the embodiments described above, but encompasses any and all embodiments within the scope of the following claims.

1. An anesthetic chewing gum, comprising a combination of an analgesic component and a chewing gum base component:
the analgesic component consists of 2 mg lidocaine HCL and 2 mg prilocaine HCL; and
the chewing gum base component comprises a chewing gum base.

2. The anesthetic chewing gum according to claim 1, wherein the chewing gum base comprises 436 mg of the chewing gum base.

3. The anesthetic chewing gum according to claim 2, further comprising:
about 30 mg of a sweetener;
about 10 mg of an anti-adherent;
about 5 mg of a lubricant;
about 5 mg of an opacifier;
about 2.5 mg of a glidant;
about 2.5 mg of a flavoring agent; and
about 5 mg of a flavor enhancer.

4. The anesthetic chewing gum according to claim 2, wherein the chewing gum is a chewing gum tablet, wherein the tablet has a diameter of about 10 mm, a thickness of about 6 mm, and a weight of about 499 mg.

5. The anesthetic chewing gum according to claim 3, wherein the chewing gum tablet has a hardness of about 4 kg/cm2.

6. The anesthetic chewing gum according to claim 3, wherein the anti-adherent comprises talc.

7. The anesthetic chewing gum according to claim 3, wherein the lubricant comprises magnesium stearate.

8. The anesthetic chewing gum according to claim 3, wherein the opacifier comprises titanium dioxide.

9. The anesthetic chewing gum according to claim 3, wherein the glidant comprises colloidal silicon dioxide.

10. The anesthetic chewing gum according to claim 3, wherein the flavoring agent comprises peppermint oil.

11. The anesthetic chewing gum according to claim 3, wherein the flavor enhancer comprises menthol.

12. A method of making an anesthetic chewing gum, the steps consisting of:
forming a plurality of separate lidocaine-HCL mixture portions by separately mixing an amount of lidocaine HCL with an amount of a secondary chewing gum ingredient, the secondary chewing gum ingredient being selected from the group consisting of a sweetener, an anti-adherent, a lubricant, an opacifier, a glidant, and a flavor enhancer;
forming a plurality of separate prilocaine HCL mixture portions by separately mixing an amount of prilocaine HCL with an amount of one of the secondary chewing gum ingredients;
mixing together each lidocaine HCL mixture portion and prilocaine HCL mixture portion having a same secondary chewing gum ingredient to form a plurality of lidocaine HCL/prilocaine HCL mixture portions;
mixing the plurality of lidocaine HCL/prilocaine HCL mixture portions together to form a homogenized lidocaine HCL-prilocaine HCL mixture;
combining the homogenized lidocaine HCL-prilocaine HCL mixture with a gum base to form a homogenized lidocaine HCL-prilocaine HCL gum base mixture;
adding a flavoring agent to the homogenized lidocaine HCL-prilocaine HCL gum base mixture to form granules;
drying the granules at room temperature;
mixing the granules to form a final granule blend; and
compressing the final granule blend into a plurality of chewing gum tablets, wherein each chewing gum tablet contains 2 mg of each of lidocaine HCL and prilocaine HCL.

13. The method of making an anesthetic chewing gum according to claim 12, wherein compressing the final granule blend comprises using a direct compression technique.

14. The method of making an anesthetic chewing gum according to claim 12, wherein the chewing gum tablets have an average diameter of about 10 mm, an average thickness of about 6 mm, and an average weight of about 499 mg.

15. The method of making an anesthetic chewing gum according to claim 12, wherein the chewing gum tablets have an average hardness of about 4 kg/cm2.

16. The anesthetic chewing gum according to claim 12, wherein the anti-adherent comprises talc.

17. The anesthetic chewing gum according to claim 12, wherein the lubricant comprises magnesium stearate.

18. The anesthetic chewing gum according to claim 12, wherein the opacifier comprises titanium dioxide.

19. The anesthetic chewing gum according to claim 12, wherein the glidant comprises colloidal silicon dioxide.

20. The anesthetic chewing gum according to claim 12, wherein the flavoring agent comprises peppermint oil.
